(19) United States
(12) Patent Application Publication
(10) Pub. No.: US 2019/0262279 A1
(43) Pub. Date: Aug. 29, 2019

(54) WATER-BASED EMOLLIENT COMPOSITIONS AND METHODS OF USE THEREFOR

(71) Applicant: Tissue Tools LLC, Conshohocken, PA (US)

(72) Inventors: Kevin Parker, Wayne, PA (US); Francis X. Osborne, West Conshohocken, PA (US)

(21) Appl. No.: 16/309,372

(22) PCT Filed: Jul. 1, 2016
(86) PCT No.: PCT/US16/40743

§ 371 Date: Dec. 12, 2018

(51) Int. Cl. A61K 31/047; A61K 9/00; A61K 47/32

(52) U.S. Cl. CPC A61K 31/047; A61K 47/32; A61K 9/0014

(57) ABSTRACT

The present application teaches a water-based skin emollient composition comprising water, a polyhydric alcohol, a diol, a thickener, and a pH-adjusting agent for use as a massage emollient during physical therapy applications, and methods of use therefor.

### FIELD OF THE INVENTION

This invention relates generally to water-based compositions for skin emollients and methods of use therefor, and more specifically to water-based compositions for use as massage emollients during physical therapy applications and methods of use therefor.

### BACKGROUND OF THE INVENTION

Many physical therapy techniques, such as manual massage therapy and instrument-assisted soft tissue mobilization (IASTM), involve direct interaction with the skin of a patient to assess and treat various soft tissue injuries and ailments. It is well understood that using a lubricating composition, such as a lotion or gel, between the therapist's hands or therapy tool and the patient's skin reduces drag and strain on the therapist's hands or tools, and allows the therapist to more effectively treat a patient.

Currently, many physical therapists and IASTM practitioners use oil-based compositions as lubricating compositions during physical therapy (PT) sessions. Oil-based compositions leave an oily residue on the patient's skin, requiring the physical therapist to clean the patient's skin with a towel, alcohol, or soap and water if they are going to apply an additional item—such as athletic tape, a bandage, an electrode, or a medicated patch—to the patient's skin after PT treatment. Oil-based compositions also stain clothes and are too thick to use in a squirt bottle without heating, which introduces an infection-control concern into their use in a clinical setting because many therapists therefor choose to apply the oil-based composition to a patient's skin from a non-sterile jar or bottle.

Further, currently some physical therapists and IASTM practitioners use ultrasound gel as a lubricating composition during PT sessions. While ultrasound gel is water-based, it is runny, gives a very short treatment time, and also lacks an ideal viscosity for the use of massage tools. Other known water-based compositions, such as personal lubricants, are not indicated or suitable for use on the skin during PT sessions.

There is a need for water-based and water-soluble emollient compositions and methods of use therefor that overcome these and other shortcomings with the known prior art.

### BRIEF DESCRIPTION OF THE DRAWINGS

The foregoing summary, as well as the following detailed description of the invention, will be better understood when read in conjunction with the appended drawings. For the purpose of illustrating the invention disclosed herein, certain embodiments in accordance with the herein disclosed invention are shown in the drawings. The present invention will hereinafter be described in conjunction with the appended drawing figures. In the drawings:

FIG. 1 is a flowchart showing the steps of a first method of treatment in accordance with the present invention; and

FIG. 2 is a flowchart showing the steps of a second method of treatment in accordance with the present invention.

### DETAILED DESCRIPTION OF THE PREFERRED EMBODIMENTS

The ensuing detailed description provides preferred exemplary embodiments only, and is not intended to limit the scope, applicability, or configuration of the herein disclosed inventions. Rather, the ensuing detailed description of the preferred exemplary embodiments will provide those skilled in the art with an enabling description for implementing the preferred exemplary embodiments in accordance with the herein disclosed invention. It should be understood that various changes may be made in the function and arrangement of elements without departing from the spirit and scope of the invention, as set forth in the appended claims.

To aid in describing the invention, directional terms may be used in the specification and claims to describe portions of the present invention (e.g., upper, lower, left, right, etc.). These directional definitions are merely intended to assist in describing and claiming the invention and are not intended to limit the invention in any way. In addition, reference numerals that are introduced in the specification in association with a drawing figure may be repeated in one or more subsequent figures without additional description in the specification in order to provide context for other features.

Unless otherwise noted, all percentages used in the specification and appended claims should be understood to refer to a per-weight basis, reflected as a percentage of the weight of the overall composition. For example, if Component A is said to be 5.00% by weight of a composition, the weight of Component A in the composition is equal to 5.00% of the weight of the entire composition.

In the specification and appended claims, the term “instrument” refers collectively to a therapist's body part that is used for physical therapy purposes, including but not limited to his or her finger, hand, arm, elbow, or knee, or a standalone article or tool that is used for the purpose of performing physical therapy on a patient, including but not limited to a piece of stone, horn, wood, glass, plastic, or metal, whether or not specifically designed for physical therapy treatment purposes.

In the specification and appended claims, the term “room temperature” refers to an environmental temperature of 70.0 degrees Fahrenheit (21.1 degrees Celsius).

Unless otherwise stated, in the specification and appended claims, the term “physical therapy” (PT) refers collectively to manual physical therapy (e.g., massage) and instrument-assisted physical therapy (e.g., IASTM).

As noted above, many physical therapists currently use an oil-based emollient on the patient's skin during PT treatment sessions. These oil-based emollients leave behind an oily residue on the patient's skin, requiring extensive cleanup—often using skin-drying alcohol—before an additional adhesive item can be applied to the patient's skin. In many cases, because a patient undergoing PT treatment has experienced a sports-related injury or other soft tissue ailment and requires further care post-therapy session, it is desirable to apply PT tape, a medicated patch, or an electrode to the patient's skin immediately after treatment. The clean-up step necessitated by the use of oil-based emollients is time-consuming and wasteful and increases the cost of PT sessions. Moreover, the oily residue that is left behind on the patient's skin can complicate the step of applying an adhesive item such as PT tape, because the adhesive on the PT tape is less likely to stick to an oily surface than a clean one.

Further, oil-based compositions are too thick to use in a squirt bottle (or other type of storage and delivery container that eliminates the need for the therapist to directly touch the patient's skin) without heating, which introduces infection-control concerns into their use in a clinical setting because many therapists therefor choose to directly apply the oil-based composition to a patient's skin from a non-sterile jar or open bottle, rather than to keep costly bottle-warming equipment around.

As noted above, some physical therapists and IASTM practitioners have attempted to use existing water-based gels, such as ultrasound gel, as a lubricating composition during PT sessions. Ultrasound gels are designed to serve as an ultrasonic medium for use with an ultrasound transducer (i.e., they are designed as a sound-transmitting medium), and are not intended for long skin-contacting periods. Because ultrasound gels are generally composed of greater than 90% water by weight, they are runny and unsuitable for extended treatment periods with an instrument. Common ultrasound gels generally have a viscosity in the range of 100,000-200,000 centipoise (cPs) at room temperature.

Other known water-based compositions, such as personal lubricants, are not indicated or ideal for use on the external skin during PT sessions. Some of these water-based compositions also generally have a high percentage by weight of water (i.e., greater than 50% by weight), and lack suitable viscosity as a gliding gel when contacted by an instrument moving over a treatment area over an extended period of time. Many of these known water-based personal lubricants have a viscosity of between 200-5,000 cPs at room temperature. These known water-based formulations will also “snowplow” or build up under or on an instrument during a PT session, thus wasting gel and requiring the therapist to pause the treatment session to clean the tools and/or requiring time-consuming instrument cleanup post-treatment.

In embodiments according to the present invention, the water-based composition preferably has a viscosity in the range of 7,500-30,000 cPs at room temperature, more preferably in the range of 10,000-17,500 cPs at room temperature, and even more preferably between 12,500-15,000 cPs at room temperature. These viscosity ranges permit the composition to be easily squeezed out of an infection-control bottle at room temperature without the need to shake the bottle or pre-heat the composition. These viscosity ranges also maintain the composition in or near the desired treatment area after application to the patient's skin, provide for an adequate treatment period without the need to reapply the composition, and minimize build-up of the composition on the therapist's instrument.

In PT treatment settings in which a patient is to be taped after receiving therapy, it may be desirable to have a first period of time following application of the emollient composition to the skin during which the coefficient of friction between the therapist's hands or tools and the patient's skin is reduced (i.e., a “glide” period), and then a second period of time following application of the emollient composition to the skin during which the coefficient of friction between the therapist's instrument and the patient's skin is increased (i.e., a “tack” period) to aid with the application of PT tape. In various embodiments according to the present invention, the first period is preferably between 2-12 minutes long, more preferably between 3-10 minutes long, and even more preferably between 5-8 minutes long; and the second period is preferably between 1-10 minutes long, more preferably between 2-7 minutes long, and even more preferably between 2-4 minutes long. These periods of time have been calculated in order to permit therapists to apply an emollient composition to the treatment area of a patient's body once, perform the PT treatment at a typical pace, and then securely apply PT tape (or some other adhesive item) to the treatment area of the patient at a typical pace, without an extensive clean-up step required between the treatment and adhesive item application steps.

In one aspect, the invention comprises a water-based skin emollient composition comprising water, a polyhydric alcohol (and/or humectant), a diol (and/or polyol), a thickener, and a pH-adjusting agent. In some embodiments according to the present invention, the water is between 25.00-50.00% by weight of the composition, and more preferably between 35.00-45.00% by weight of the composition. In some embodiments according to the present invention, the polyhydric alcohol (or humectant) is between 5.00-50.00% by weight of the composition, and more preferably between 15.00-30.00% by weight of the composition. In some embodiments according to the present invention, the diol (or polyol) is between 5.00-50.00% by weight of the composition, and more preferably between 15.00-30.00% by weight of the composition. In some embodiments according to the present invention, the thickener is between 0.01-0.50% by weight of the composition, and more preferably between 0.025-0.20% by weight of the composition. In some embodiments according to the present invention, the pH-adjusting agent is between 0.01-0.50% by weight of the composition, and more preferably between 0.025-0.20% by weight of the composition.

In various embodiments according to the present invention the water is purified or deionized water; although in the alternative any type of water could be used. In various embodiments according to the present invention, suitable polyhydric alcohols (sugar alcohols) include, but are not limited to: glycerin (glycerol), erythritol, threitol, arabitol, xylitol, ribitol, mannitol, sorbitol, galactitol, fucitol, iditol, inositol, volemitol, isomalt, maltitol, lactitol, maltotriitol, maltotetraitol, and polyglycitol, or any combination thereof. In various embodiments according to the present invention, the polyhydric alcohol in the composition may be replaced with a suitable humectant, including but not limited to: sodium PCA, honey, aloe vera gel, nanolipid gels, urea, or any combination thereof.

In various embodiments according to the present invention, suitable diols include, but are not limited to: ethylene glycol, butylene glycol, propylene glycol, propolynese glycol, diethylene glycol, triethylene glycol, polyalkylene glycols, polyethylene glycol, 1,2-propanediol, 1,3-propanediol, butanediol(1,3), butanediol(1,4), hexanediol(1,6), and isomers thereof, neopentyl glycol or hydroxypivalic neopentyl glycol ester, hexanediol(1,6) and isomers thereof, butanediol(1,4), neopentyl glycol, and hydroxypivalic neopentyl glycol ester, or any combination thereof. The use of propanediol as the diol in the composition will boost the efficacy of any preservatives used in the composition, therefore permitting a reduction in the amount of preservative used. In alternate embodiments according to the present invention, the diol could be replaced with a polyol, including but not limited to: trimethylolpropane, erythritol, pentaerythritol, trimethylolbenzene, trishydroxyethyl isocyanurate, or any combination thereof.

In various embodiments according to the present invention, suitable thickeners include, but are not limited to: any carbomer, for example any cross-linked homopolymer of acrylic acid such as Carbopol Ultrez 30 Polymer produced by Lubrizol Advanced Materials, Inc. of Cleveland, Ohio, U.S.A., which has the chemical structure depicted below:

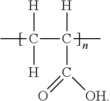

Suitable thickeners also include, but are not limited to: various gums, including but not limited to xanthum gum, guar gum, agar agar, carrageenan, acacia, locust bean, and any combination thereof; and various celluloses, including but not limited to: hydroxyethyl cellulose, methyl cellulose, hydroxypropyl methyl cellulose, and any combination thereof.

In some embodiments according to the present invention, it is preferable for the composition to have a pH in the range of 6.0-7.0. If an acidic carbomer is used as the thickener, the pH of the composition will be significantly lowered, therefore requiring the addition of a pH-adjusting agent to bring the pH back up into the desired range. In these embodiments, suitable pH-adjusting agents include, but are not limited to: aminomethyl propanol (AMP), sodium hydroxide, triethylamine (TEA), ammonium hydroxide, potassium hydroxide, triethanolamine, tromethamine, and any combination thereof. If a gum or cellulose is used as the thickener in the composition, it may be possible to reduce or eliminate altogether the need to add a pH-adjusting agent to the composition, owing to the relatively neutral pH levels of these ingredients. In embodiments in which a gum or cellulose is used as the thickener, the thickener may comprise between 0.01-2.00% by weight of the composition and the pH-adjusting agent could be omitted entirely or comprise up to 2.00% by weight of the composition.

In various embodiments according to the present invention, the composition may further comprise a surfactant that adds to the “glideability” of the composition. In these embodiments, suitable surfactants include, but are not limited to: polyethylene glycol laurate (PEG), for example PEG-8 (PEG-400) produced by Acme Hardesty Company of Blue Bell, Pa., U.S.A., any other PEGs, any PEG-containing compounds, any polyethylene oxides (PEOs), PEO-containing compounds, and any combination thereof. In some embodiments according to the present invention, the surfactant comprises between 0.50-10.00% by weight of the composition, and more preferably between 2.50-7.50% by weight of the composition.

In various embodiments according to the present invention, the composition may further comprise one or more suitable preservatives. In these embodiments, suitable preservative(s) include, but are not limited to: phenoxyethanol; ethylhexyglycerin; parabens including but not limited to Germaben II, methylparaben, propylparaben, butylparaben, and combinations thereof formaldehyde releasers including but not limited to German® Plus, sold by Ashland Inc. of Covington, Ky., U.S.A., DMDH hydantoin, imadozolidinyl urea, diazolidinyl urea, and combinations thereof; isothiazolinones including but not limited to Kathon®, sold by The Dow Chemical Company of Midland, Mich., U.S.A.; organic acids including but not limited to benzoic acid/sodium benzoate, sorbic acid/potassium sorbate, levulinic acid, anisic acid, citric acid, lactic acid, L-ascorbic acid, and combinations thereof; and combinations of any of the foregoing. In some preferred embodiments, the preservative is a combination of phenoxyethanol and ethylhexyglycerin that is sold under the name Euxyl® PE 9010 by Schülke & Mayr GmbH of Norderstedt, Germany. In some embodiments according to the present invention, the preservative comprises between 0.50-1.00% by weight of the composition, and more preferably between 0.65-0.85% by weight of the composition.

In various embodiments according to the present invention, an antiseptic component may be added to the composition, to further assist with infection control. Suitable antiseptic components for inclusion in any embodiment according to the present invention include, but are not limited to: alcohols, including ethyl alcohol 70% and isopropyl alcohol 70%; quaternary ammonium compounds, including benzalkonium chloride, cetrimide, methylbenzethonium chloride, benzethonium chloride, cetalkonium chloride, cetylpyridinium chloride, dofanium chloride, and domiphen bromide; chlorhexidine and other diguanides, including chlorhexidine gluconate and chlorhexidine acetate; antibacterial dyes, including proflavine hemisulphate, triphenylmethane, brilliant green, crystal violet, and gentian violet; peroxides and permanganates, including hydrogen peroxide solution, potassium permanganate solution, and benzoyl peroxide; halogenated phenol derivatives, including chlorocresol, chloroxylenol, chlorophene, hexachlorophane/hexachlorophene, and triclosan; quinolone derivatives, including hydroxyquinoline sulphate, potassium hydroxyquinoline sulphate, chlorquinaldol, dequalinium chloride, and di-iodohydroxyquinoline; other components such as Burow's solution (an aqueous solution of aluminum acetate) and bleach baths; and combinations of any of the foregoing.

In various embodiments according to the present invention, the composition may further comprise a coloring agent, fragrance, essential oil, botanical extract, and/or at least one additional component that imparts a heating or cooling effect to the skin or acts as a topical pain relief component (i.e., an analgesic component), including but not limited to arnica, menthol, camphor, capsaicin, magnesium, green tea oil, white tea oil, lavender oil, and any combination thereof.

In another aspect, the present invention comprises an additional water-based emollient composition in accordance with the aspect set forth above, but further comprising an adhesive component that generates a “tacky” period on the patient's skin following an initial “slippery” period following application of the emollient to the patient's skin. This “tacky” period—in which the coefficient of friction of the patient's skin is greater than normal—can assist with the adhesion of PT tape, a bandage, an electrode, a medicated patch, or some other adhesive article to the patient's skin after PT treatment. In these embodiments, suitable adhesive components include, but are not limited to: a high-molecular weight polyvinylpyrrollidone (PVP) with various K values, for example those sold under the brand name Luvitec® by BASF Corporation of Ludwigshafen, Germany; lower-molecular weight PVPs with various K values; a combination of PVP and vinyl acetate (PVP/VA); or any combination thereof. In some embodiments according to the present invention, the adhesive component comprises between 0.50-10.00% by weight of the composition, and more preferably between 2.50-7.50% by weight of the composition. PVP is a water-soluble polymer that will remain slippery while in an aqueous solution but will become tacky as the water evaporates, thus providing the desired effects of this embodiment.

In yet another aspect, the invention comprises an anhydrous (i.e., waterless) emollient composition comprising a polyhydric alcohol (or humectant) comprising between 5.00-75.00% by weight of the composition, a diol (or polyol) comprising between 5.00-75.00% by weight of the composition, a thickener comprising between 0.01-0.50% by weight of the composition, and a pH-adjusting agent comprising between 0.01-0.50% by weight of the composition. The precise ingredients used as the polyhydric alcohol, diol, thickener, and pH-adjusting agent in these embodiments may be chosen from the lists of suitable ingredients set forth above with respect to the water-containing emollient aspect of the present invention. The polyhydric alcohol may comprise more preferably between 25.00-50.00% by weight of the composition, the diol may comprise more preferably between 25.00-50.00% by weight of the composition, the thickener may comprise more preferably between 0.025-0.20% by weight of the composition, and the pH-adjusting agent may comprise more preferably between 0.025-0.20% by weight of the composition. In alternate embodiments according to the present invention, if a gum or cellulose is used as the thickener, the thickener comprises between 0.01%-2.00% by weight of the composition and the pH-adjusting agent could be omitted entirely or form up to 2.00% by weight of the composition.

An anhydrous emollient composition is believed to resist contamination better than a water-based emollient composition, and is believed to be stable enough to permit the omission of a preservative from the composition. An anhydrous emollient composition, or a composition in which there is significantly more glycerin (or other polyhydric alcohol) than water, would tend to stay longer on a patient's skin and would therefore be particularly well-suited to long therapy sessions (e.g., manual massage therapy sessions). In various embodiments of this aspect of the present invention, the anhydrous emollient composition may further comprise any of the surfactant, preservative, antiseptic, or additional ingredients set forth above with respect to the water-based emollient aspect of the present invention.

The water-based emollients according to the present invention have the following benefits over known oil-based emollients and water-based gels such as ultrasound gel and personal lubricants: they will not “snowplow” under instruments (e.g., tools) during use; they are easy to clean off of the skin without the need for alcohol or soap; they are less likely to run onto or stain clothing; they permit for the addition of an adhesive element that may aid with application of an adhesive article after the therapy session by providing a later-occurring “tacky” period (certainly an undesirable feature in the case of ultrasound gels and personal lubricants); their viscosities and material characteristics are appropriate for PT applications, thereby permitting the use of far less emollient on the patient's skin in comparison to, for example, ultrasound gel, thus resulting in significant cost savings; and their viscosities permit their storage in and dispensation from infection-controlled containers (e.g., squirt-bottles), which is not possible with oil-based emollients absent the use of expensive bottle-heating equipment.

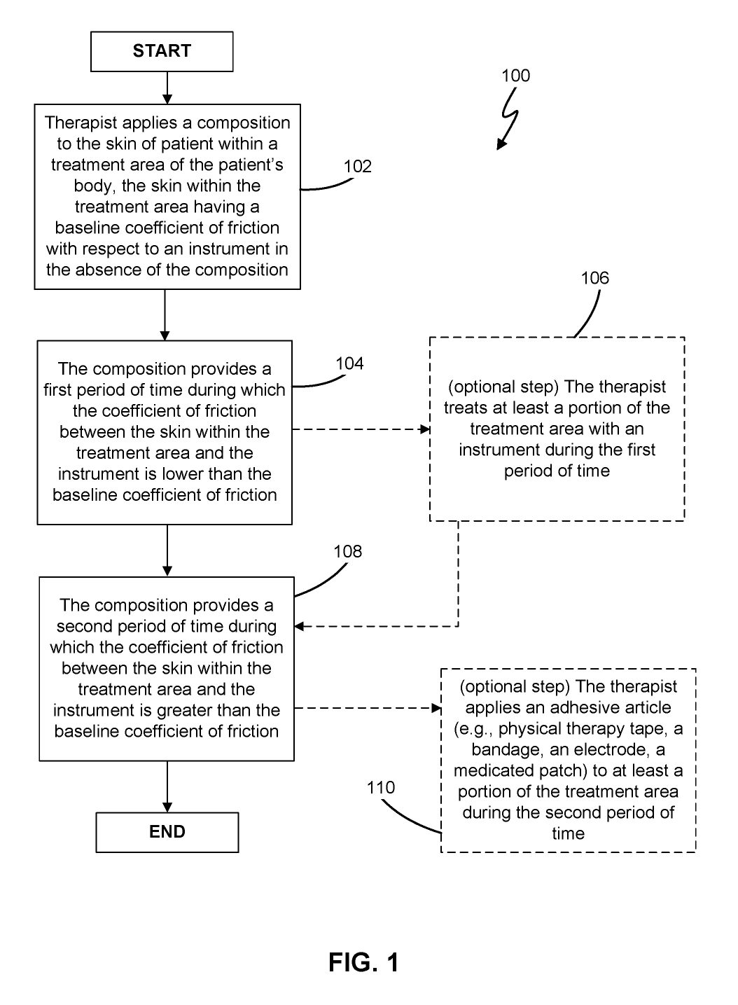

In a still further aspect, as shown in the flowchart of FIG. 1, the present invention comprises a method 100 of applying an emollient composition to the skin of a patient associated with a treatment area on the patient's body, the skin within the treatment area having a baseline coefficient of friction (with respect to an instrument) that is measured in the absence of (i.e., prior to application of) the emollient composition (step 102), the emollient composition providing a first period of time during which the coefficient of friction (with respect to the instrument) within the treatment area is reduced relative to the baseline coefficient of friction (i.e., a “glide” period during which an instrument is typically used to treat the treatment area of the patient's body; step 104), the emollient composition further providing a second period of time following the first period of time during which the coefficient of friction (with respect to the instrument) within the treatment area is increased relative to the baseline coefficient of friction (i.e., a post-treatment “tack” period that may aid in the application of PT tape or other accessories; step 108). In various embodiments according to the present invention, the first period is preferably between 2-12 minutes long, more preferably between 3-10 minutes long, and even more preferably between 5-8 minutes long; and the second period is preferably between 1-10 minutes long, more preferably between 2-7 minutes long, and even more preferably between 2-4 minutes long. After applying the emollient composition to the skin within the treatment area of the patient at step 102, the therapist may optionally treat at least a portion of the treatment area with an instrument (e.g., hands or tools) during the first period (step 106) and/or optionally apply an adhesive article (e.g., physical therapy tape, a bandage, an electrode, or a medicated patch) to at least a portion of the treatment area during the second period (step 110). In some embodiments according to this aspect, the composition has a viscosity between 7,500-30,000 centipoise at room temperature, and more preferably between 10,000-17,500 centipoise at room temperature.

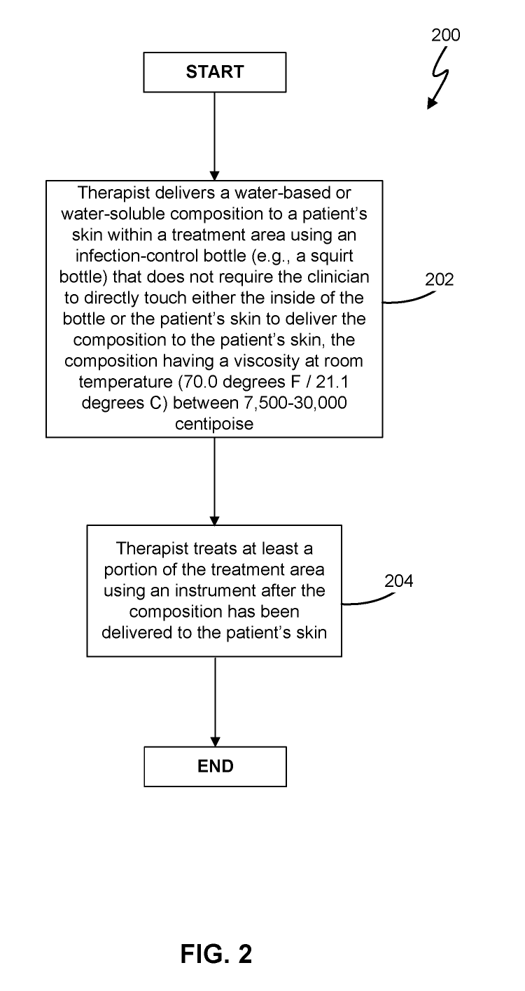

In a still further aspect, as shown in the flowchart of FIG. 2, the present invention comprises a method 200 of, in a first step 202, delivering a water-based or water-soluble composition to a patient's skin within a desired treatment area using an infection-control bottle (e.g., a squirt bottle) that does not require the clinician to directly touch either the inside of the bottle or the patient's skin, the composition having a viscosity at room temperature between 7,500-30,000 cPs, and then, in a second step 204, conducting a PT or IASTM treatment session on at least a portion of the patient's treatment area using an instrument (e.g., the therapist's hands or a physical tool). In this way, the composition has been provided with a specialized viscosity range that permits it to be squeezed out of an infection-control bottle at room temperature (without the need to pre-heat the composition or shake the bottle), and then perform well as an emollient for physical therapy sessions. In some embodiments according to this aspect, the composition more preferably has a viscosity between 10,000-17,500 centipoise at room temperature.

### EXAMPLES

The invention will now be further described with the help of the following non-limiting examples.

Base formulas 1 and 2 (components shown in % w/w) (percentages may not total exactly to 100.00% due to rounding):

| Component | Formula 1 | Formula 2 |
|---|---|---|
| Purified Water | 44.05 | 42.48 |
| Propanediol | 25.00 | 22.00 |
| Glycerin | 25.00 | 26.00 |
| PEG-8 (polyethylene glycol laurate) | 5.00 | 3.70 |
| Phenyoxyethanol & ethylhexyglycerin | 0.75 | 0.75 |
| Carbomer | 0.13 | 0.08 |
| Aminomethyl propanol | 0.07 | 0.05 |
| High-molecular weight polyvinylpyrrollidone | 0.00 | 5.00 |
| (PVP) | | |

It should be appreciated that the foregoing is presented by way of illustration only, and not by way of any limitation, and that various alternatives and modifications may be made to the illustrated embodiments without departing from the spirit and scope of the present invention.

1. A water-based skin emollient composition, the composition comprising:
water, the water comprising between 25.00-50.00% by weight of the composition;
a polyhydric alcohol, the polyhydric alcohol comprising between 5.00-50.00% by weight of the composition;
a diol, the diol comprising between 5.00-50.00% by weight of the composition;
a thickener, the thickener comprising between 0.01-0.50% by weight of the composition; and
a pH-adjusting agent, the pH-adjusting agent comprising between 0.01-0.50% by weight of the composition.

2. The composition according to claim 1, further comprising a surfactant, the surfactant comprising between 0.50-10.00% by weight of the composition.

3. The composition according to claim 2, wherein the surfactant is polyethylene glycol laurate.

4. (canceled)

5. The composition according to claim 1, further comprising at least one preservative, the at least one preservative comprising between 0.50-1.00% by weight of the composition.

6. (canceled)

7. The composition according to claim 1, wherein the diol is propanediol.

8. The composition according to claim 1, wherein the water comprises between 35.00-45.00% by weight of the composition, the polyhydric alcohol comprises between 15.00-30.00% by weight of the composition, and the diol comprises between 15.00-30.00% by weight of the composition.

9. The composition according to claim 1, wherein the thickener comprises between 0.025-0.20% by weight of the composition.

10. The composition according to claim 1, wherein the percentage by weight of water in the composition is at least 50% greater than the percentage by weight of the polyhydric alcohol in the composition.

11. The composition according to claim 1, wherein the percentage by weight of the polyhydric alcohol in the composition is greater than the percentage by weight of the water in the composition.

12. The composition according to claim 1, further comprising an adhesive component.

13. The composition according to claim 12, wherein the adhesive component comprises between 0.50-10.00% by weight of the composition.

14. The composition according to claim 12, wherein the adhesive component is a high-molecular weight polyvinylpyrrollidone.

15. (canceled)

16. (canceled)

17. (canceled)

18. The composition according to claim 1, wherein the composition has a viscosity between 10,000-17,500 centipoise at room temperature.

19. The composition according to claim 1, wherein the polyhydric alcohol is replaced with a humectant in the same percentage by weight of the composition.

20. (canceled)

21. A water-soluble skin emollient composition, the composition comprising:
a polyhydric alcohol, the polyhydric alcohol comprising between 5.00-75.00% by weight of the composition;
a diol, the diol comprising between 5.00-75.00% by weight of the composition;
a thickener, the thickener comprising between 0.01-0.50% by weight of the composition; and
a pH-adjusting agent, the pH-adjusting agent comprising between 0.01-0.50% by weight of the composition.

22. The composition according to claim 21, wherein the polyhydric alcohol comprises between 25.00-50.00% by weight of the composition and the diol comprises between 25.00-50.00% by weight of the composition.

23. (canceled)

24. The composition according to claim 21, wherein the composition has a viscosity between 10,000-17,500 centipoise at room temperature.

25. A method comprising:
applying a composition to the skin of a patient within a treatment area of the patient's body, the skin within the treatment area having a baseline coefficient of friction that is measured in the absence of the composition, the composition providing a first period of time during which a first measured coefficient of friction of the skin within the treatment area is less than the baseline coefficient of friction, the composition further providing a second period of time following the first period of time during which a second measured coefficient of friction of the skin within the treatment area is greater than the baseline coefficient of friction.

26.-29. (canceled)

30. The method of claim 25, wherein the composition is the composition according to claim 1.

31.-34. (canceled)

35. The method of claim 25, wherein the composition is the composition according to claim 21.
